(19) United States
(12) Patent Application Publication
(10) Pub. No.: US 2025/0383147 A1
(43) Pub. Date: Dec. 18, 2025

(54) DRYING DEVICE FOR VEHICLES AND VEHICLE EQUIPPED WITH THE SAME

(71) Applicant: HYUNDAI MOBIS CO., LTD., Seoul (KR)

(72) Inventors: Sung Joon AHN, Yongin-si (KR); Ji Soo SHIN, Yongin-si (KR); Tae Hun KIM, Yongin-si (KR); Seung Jae KIM, Yongin-si (KR)

(73) Assignee: HYUNDAI MOBIS CO., LTD., Seoul (KR)

(21) Appl. No.: 18/976,993

(22) Filed: Dec. 11, 2024

(30) Foreign Application Priority Data
Jun. 14, 2024 (KR) 10-2024-0077532

(51) Int. Cl. F26B 9/00; A61L 2/04; A61L 2/10; B60H 3/00; B60H 3/02

(52) U.S. Cl. CPC F26B 9/003; A61L 2/04; A61L 2/10; B60H 3/0078; B60H 3/024; A61L 2202/122; A61L 2202/26

(57) ABSTRACT

A drying device for vehicles and a vehicle therefor are provided. The drying device includes an air conditioner for exhaust and intake, and a sterilization dryer connected to the air conditioner and having formed therein a storage space. The sterilization dryer includes an intake line to direct air exhausted from the air conditioner into the storage space, a discharge line to externally discharge air that has flowed through the intake line and passed through the storage space, and an exhaust line to direct air that has flowed through the intake line and passed through the storage space, into the air conditioner.

### CROSS-REFERENCE TO RELATED APPLICATION(S)

This application claims the benefit under 35 USC § 119 of Korean Patent Application No. 10-2024-0077532 filed on Jun. 14, 2024, in the Korean Intellectual Property Office, the entire disclosure which is hereby incorporated by reference for all purposes.

### BACKGROUND

### 1. Field

The present disclosure relates to a drying device for vehicles capable of sterilizing, drying, and deodorizing clothing or shoes inside a vehicle based on an air conditioning function of the vehicle and a vehicle equipped with the same.

### 2. Description of the Related Art

Recently, due to rapid improvement in the quality of life and increasing demands therefor, various convenience devices are being installed in vehicles. In particular, since the interior space of a vehicle functions as another living space, the demand for the installation of various convenience devices is becoming even stronger.

For example, the air conditioner sterilization and bacterial growth prevention function installed in the vehicle not only completely removes moisture generated by the air conditioner but also eliminates various foreign substances that may adhere to the air conditioner filter, thereby creating a more comfortable indoor environment in the vehicle.

Recently, environmental pollution has been worsening, and fine dust including ultrafine dust has also been increasing. As a result, when boarding the vehicle after outdoor activities, fine dust and other foreign substances that remain on clothing or shoes may become an issue.

Thus, occupants may demand functions such as sterilization or drying for their clothing or shoes. However, there are currently no convenience devices in the vehicle to meet the need, and even if similar functions exist, the effectiveness thereof is minimal.

Hence, there is a need for a means capable of efficiently using the interior space of the vehicle to store clothing or shoes and also effectively sterilizing, drying, and deodorizing the clothing or shoes to address the aforementioned issues.

### SUMMARY

The present disclosure aims to provide a drying device for vehicles and a vehicle equipped with the same. More specifically, the present disclosure aims to provide a drying device for vehicles capable of sterilizing, drying, and deodorizing clothing or shoes inside a vehicle based on an air conditioning function of the vehicle and a vehicle equipped with the same.

Additionally, the present disclosure aims to provide a drying device for vehicles and a vehicle equipped with the same, which enhances space utilization within the vehicle and allows for efficient storage and management of clothing or shoes of occupants.

It will be appreciated by persons skilled in the art that the objects that could be achieved with the present disclosure are not limited to what has been particularly described hereinabove and the above and other objects that the present disclosure could achieve will be more clearly understood from the following detailed description.

In a general aspect of the disclosure, a drying device for a vehicle, includes: an air conditioner configured for exhaust and intake; and a sterilization dryer connected to the air conditioner, the sterilization dryer connected to the air conditioner and having formed therein a storage space, wherein the sterilization dryer includes: an intake line configured to direct air exhausted from the air conditioner into the storage space; a discharge line configured to externally discharge air that has flowed through the intake line and passed through the storage space; and an exhaust line configured to direct air that has flowed through the intake line and passed through the storage space, into the air conditioner.

At least one of the discharge line or the exhaust line may be opened and closed to move the air that has passed through the storage space.

The sterilization dryer may include: a heating member configured to heat the storage space; and an ultraviolet (UV) light emitting diode (LED) configured to emit UV light into the storage space.

The heating member may include at least one of a heating coil configured to generate heat based on application of an electric current or a steam generator configured to generate heat by heating fluid.

The sterilization dryer may include an antimicrobial sheet configured to be detachable from the intake line.

The sterilization dryer may include a dust collection filter configured to be detachable from at least one of the intake line or the storage space.

The sterilization dryer may include a drain line formed at a bottom of the storage space and configured to drain moisture.

The sterilization dryer may be installed in at least one of a floor panel, a seat, or a trunk, wherein a size of the storage space may vary depending on the installation location.

The air conditioner may include: an exhaust hole connected to the intake line and configured to be opened and closed; an intake hole connected to the exhaust line and configured to be opened and closed; and an opening/closing member configured to control opening and closing of the exhaust hole and the intake hole.

In another general aspect of the disclosure, a vehicle includes: a user input unit configured to receive an input from an occupant of the vehicle; a drying device installed in a body of the vehicle; and a processor configured to operate the drying device based on the received input from the occupant, wherein the drying device includes: an air conditioner configured for exhaust and intake; and a sterilization dryer connected to the air conditioner, the sterilization dryer including a storage space, wherein the sterilization dryer includes: an intake line configured to direct air exhausted from the air conditioner into the storage space; a discharge line configured to externally discharge air that has flowed through the intake line and passed through the storage space; and an exhaust line configured to direct air that has flowed through the intake line and passed through the storage space, into the air conditioner.

The sterilization dryer may further include: a heating member configured to heat the storage space; and an ultraviolet (UV) light emitting diode (LED) configured to emit UV light into the storage space and disinfect.

The heating member may include a heating coil.

The sterilization dryer may include an antimicrobial sheet configured to be detachable from the intake line.

The sterilization dryer may include a dust collection filter configured to be detachable from at least one of the intake line or the storage space.

The sterilization dryer may be configured to be installed in at least one of a floor panel, a seat, or a trunk of the vehicle.

As is apparent from the above description, the present disclosure has effects as follows.

The drying device for vehicles and the vehicle equipped with the same according to the present disclosure may sterilize, dry, and deodorize clothing or shoes inside the vehicle based on an air conditioning function of the vehicle.

Additionally, the drying device for vehicles and the vehicle equipped with the same may enhance space utilization within the vehicle and allow for efficient storage and management of clothing or shoes of occupants.

The effects obtainable from the present disclosure are not limited to those mentioned above. Other effects not explicitly mentioned will be clearly understood by those skilled in the art from the following description.

### BRIEF DESCRIPTION OF THE DRAWINGS

The accompanying drawings, which are included to provide a further understanding of the disclosure and are incorporated in and constitute a part of this application, illustrate embodiment(s) of the disclosure and together with the description serve to explain the principle of the disclosure. In the drawings:

FIG. 1 is a block diagram of a vehicle according to an embodiment of the present disclosure;

FIG. 2 is a block diagram of a drying device for vehicles according to an embodiment of the present disclosure;

FIG. 3 is a perspective view of an air conditioner of the drying device for vehicles according to an embodiment of the present disclosure;

FIG. 4 is a side view of the air conditioner of the drying device for vehicles according to an embodiment of the present disclosure;

FIG. 5 is a cross-sectional view along line A-A of FIG. 3;

FIG. 6 is a diagram illustrating a sterilization dryer of the drying device for vehicles according to an embodiment of the present disclosure; and

FIG. 7 is a diagram for explaining airflow through the air conditioner and the sterilization dryer in the drying device for vehicles according to an embodiment of the present disclosure.

FIG. 8 is a diagram illustrating area B of FIG. 6.

### DETAILED DESCRIPTION

Description will now be given in detail according to exemplary embodiments disclosed herein, with reference to the accompanying drawings. The same or equivalent components may be provided with the same reference numbers, and description thereof will not be repeated. As used herein, the suffixes “module” and “part” are added or used interchangeably to facilitate preparation of this specification and are not intended to suggest distinct meanings or functions. In describing embodiments disclosed in this specification, relevant well-known technologies may not be described in detail in order not to obscure the subject matter of the embodiments disclosed in this specification. In addition, it should be noted that the accompanying drawings are only for easy understanding of the embodiments disclosed in the present specification, and should not be construed as limiting the technical spirit disclosed in the present specification. As such, the present disclosure should be construed to extend to any alterations, equivalents, and substitutes in addition to those which are particularly set out in the accompanying drawings.

Although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, it will be understood that when an element is referred to as being “directly connected with” another element, there are no intervening elements present.

A singular representation may include a plural representation unless it represents a definitely different meaning from the context.

The terms such as “include” or “have” used herein are intended to indicate that features, numbers, steps, operations, elements, components, or combinations thereof used in the following description exist and it should be thus understood that the possibility of existence or addition of one or more different features, numbers, steps, operations, elements, components, or combinations thereof is not excluded.

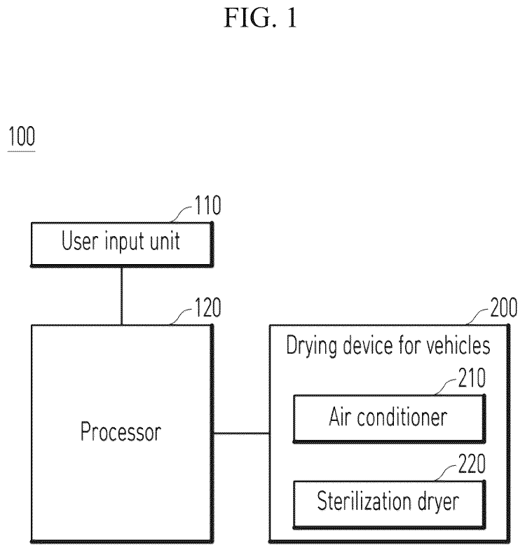

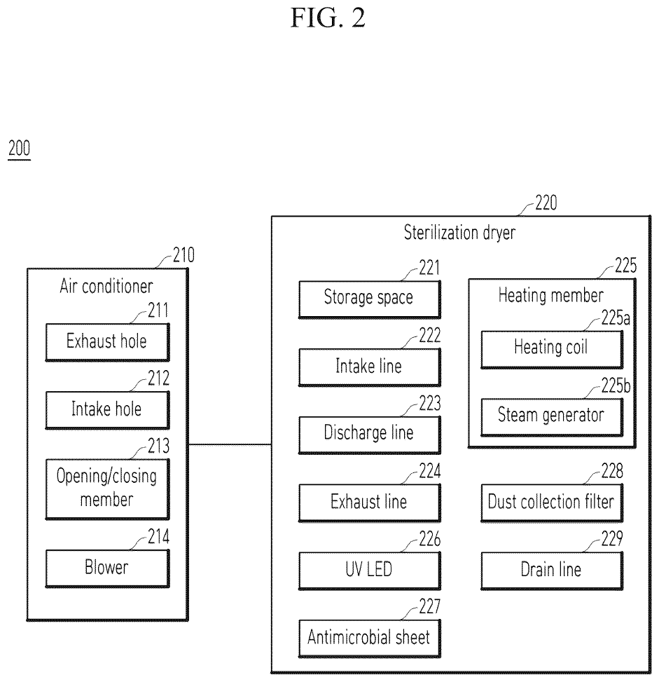

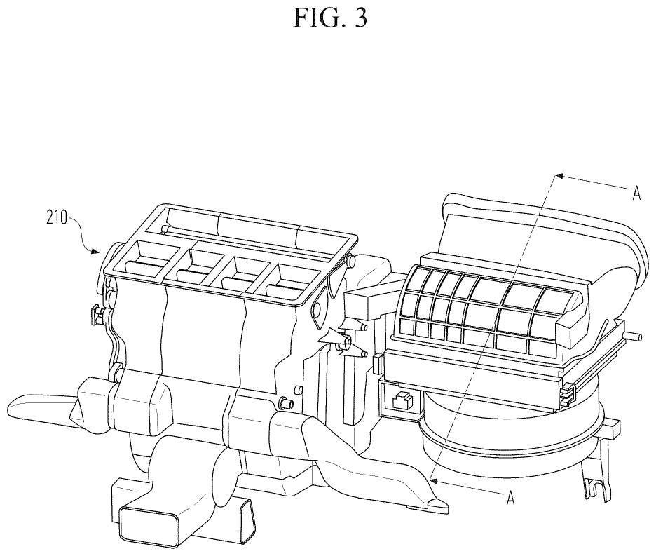

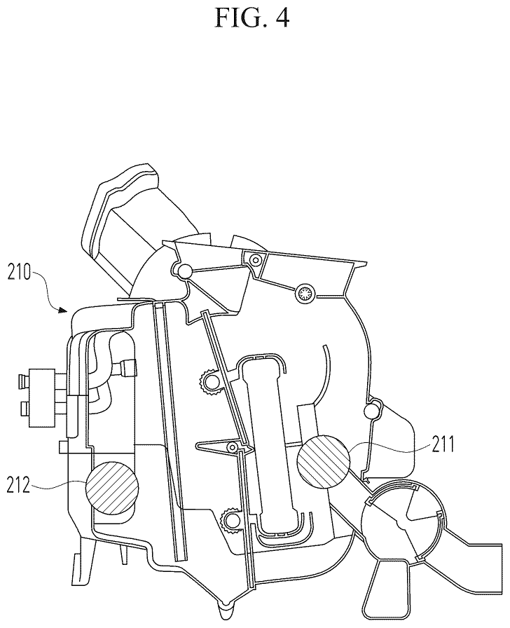

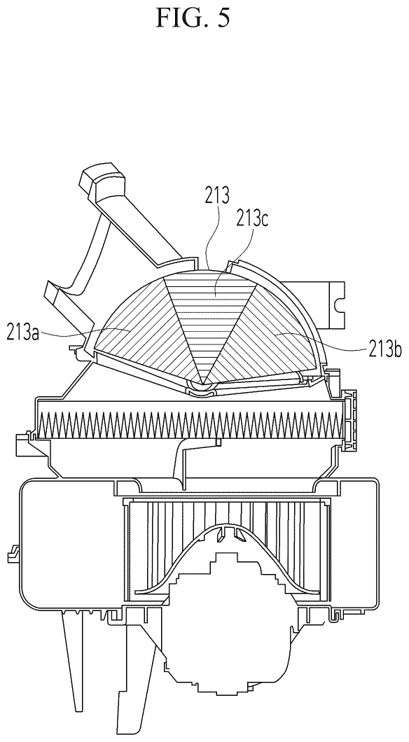

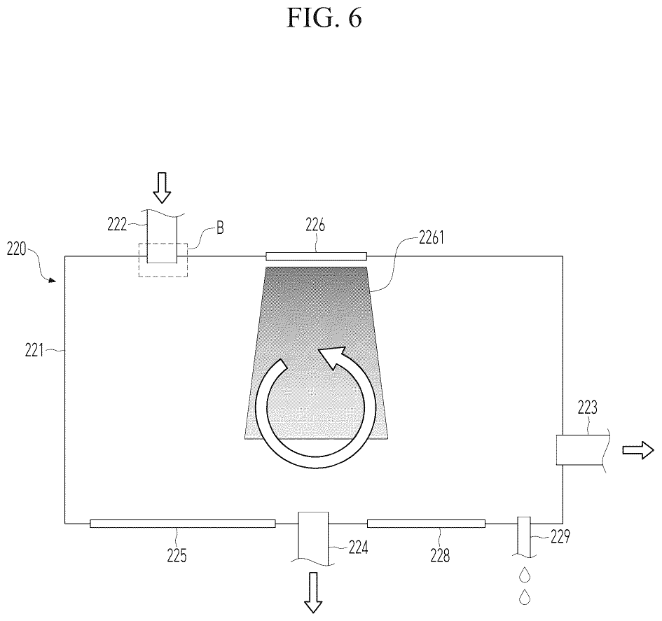

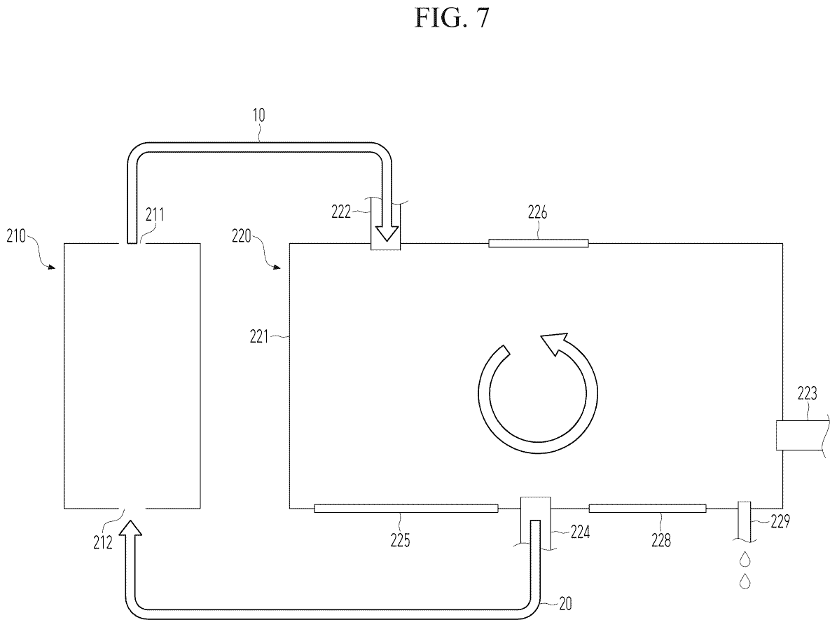

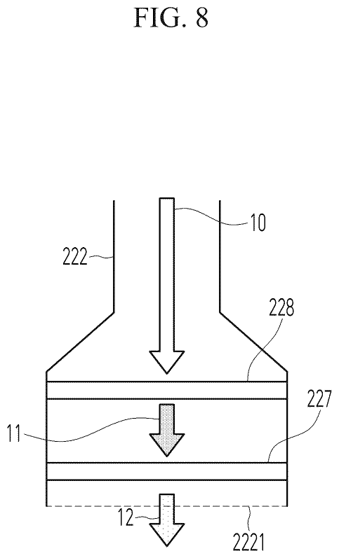

FIG. 1 is a block diagram of a vehicle 100 according to an embodiment of the present disclosure. FIG. 2 is a block diagram of a drying device for vehicles 200 according to an embodiment of the present disclosure. FIG. 3 is a perspective view of an air conditioner 210 of the drying device for vehicles 200 according to an embodiment of the present disclosure. FIG. 4 is a side view of the air conditioner 210 of the drying device for vehicles 200 according to an embodiment of the present disclosure. FIG. 5 is a cross-sectional view along line A-A of FIG. 3. FIG. 6 is a diagram illustrating a sterilization dryer 220 of the drying device for vehicles 200 according to an embodiment of the present disclosure. FIG. 7 is a diagram for explaining airflow through the air conditioner 210 and the sterilization dryer 220 in the drying device for vehicles 200 according to an embodiment of the present disclosure. FIG. 8 is a diagram illustrating area B of FIG. 6.

First, referring to FIG. 1 and FIG. 2, the vehicle 100 according to the embodiment of the present disclosure may include a user input unit 110, the drying device for vehicles 200, and a processor 120. Here, the user input unit 110 may serve to receive an input from an occupant. The drying device for vehicles 200 may be installed in a vehicle body and may include the air conditioner 210 and the sterilization dryer 220, which will be described later. Additionally, the processor 120 may operate the drying device for vehicles 200 based on the input received from the occupant via the user input unit 110.

The drying device for vehicles 200 according to the embodiment of the present disclosure may include: the air conditioner 210 capable of exhaust and intake; and the sterilization dryer 220 connected to the air conditioner 210 and having a storage space 221 formed therein. The drying device for vehicles 200 according to the embodiment of the present disclosure may use the air conditioning function of the vehicle 100 to sterilize, dry, and deodorize the clothing or shoes of the occupant stored in the storage space 221.

To sterilize, dry, and deodorize clothing or shoes of various sizes, the drying device for vehicles 200 may be installed in at least one of a floor panel, a seat, or a trunk. The size of the storage space 221 may be adjusted depending on a location where the drying device for vehicles 200 is installed.

The air conditioner 210 in the drying device for vehicles 200 according to an embodiment of the present disclosure may include an exhaust hole 211, an intake hole 212, an opening/closing member 213, and a blower 214. The sterilization dryer 220 may include the storage space 221, an intake line 222, a discharge line 223, an exhaust line 224, a heating member 225, an ultraviolet light emitting diode (UV LED) 226, an antimicrobial sheet 227, a dust collection filter 228, and a drain line 229.

Referring to FIGS. 3 to 8, the intake line 222 of the sterilization dryer 220 may serve to allow the air exhausted from the air conditioner 210 to flow into the storage space 221. The intake line 222 may be connected to the exhaust hole 211 of the air conditioner 210.

The exhaust line 224 of the sterilization dryer 220 may serve to intake the air that flows through the intake line 222 and passes through the storage space 221 back into the air conditioner 210. The exhaust line 224 may be connected to the intake hole 212 of the air conditioner 210.

The discharge line 223 of the sterilization dryer 220 may serve to discharge the air that flows through the intake line 222 and passes through the storage space 221 to the outside. Herein, the “outside” may refer to the exterior of the vehicle 100.

In other words, the drying device for vehicles 200 according to the embodiment of the present disclosure may use the air conditioner 210 to circulate air through the storage space 221, where the clothing or shoes of the occupant are stored, thereby allowing the clothing or shoes stored in the storage space 221 to be dried.

In the drying device for vehicles 200 according to the embodiment of the present disclosure, the sterilization dryer 220 may include the heating member 225 for heating the storage space 221 and the UV LED 226 for emitting UV light 2261 onto the storage space 221. Accordingly, the clothing or shoes stored in the storage space 221 may be sterilized and dried. In other words, to sterilize the clothing or shoes, the drying device for vehicles 200 of the present disclosure may heat the storage space 221 via the heating member 225 and also emit the UV light 2261 through the UV LED 226.

In the drying device for vehicles 200 according to the embodiment of the present disclosure, the sterilization dryer 220 may include the antimicrobial sheet 227 detachable from the intake line 222. The sterilization dryer 220 may also include the dust collection filter 228 detachable from at least one of the intake line 222 or the storage space 221. Accordingly, the clothing or shoes stored in the storage space 221 may be not only sterilized and dried but also deodorized.

For example, as shown in FIG. 8, foreign substances may be filtered from air 10 exhausted from the air conditioner 210 by the dust collection filter 228 detachable from the intake line 222. Air 11 that passes through the dust collection filter 228 may then pass through the antimicrobial sheet 227, and air 12 that passes through the antimicrobial sheet 227 may flow into the storage space 221. Additionally, the intake line 222 may include a backflow prevention member 2221 to prevent the air that flows into the storage space 221 from moving back into the intake line 222.

Additionally, as shown in FIGS. 6 and 7, the dust collection filter 228 may be positioned at the bottom of the storage space 221 to allow foreign substances removed from the clothing or shoes stored in the storage space 221 to be collected by the dust collection filter 228. In this case, since the occupant may easily replace the detachable dust collection filter 228, the storage space 221 of the sterilization dryer 220 can be maintained in a clean and comfortable condition.

Additionally, in the drying device for vehicles 200 according to the embodiment of the present disclosure, the sterilization dryer 220 may include the heating member 225 for heating heat the storage space 221 as described above. The heating member 225 may be a heating coil 225a that generates heat when an electric current is applied. Alternatively, the heating member 225 may be a steam generator 225b that heats fluid to produce steam.

By heating the storage space 221 through the heating member 225, the air that flows into the storage space 221 may be heated, thereby allowing the clothing or shoes of the occupant stored in the storage space 221 to be dried. In addition, the clothing or shoes of the occupant may be sterilized through heat or steam generated by the heating member 225.

In the drying device for vehicles 200 according to the embodiment of the present disclosure, the sterilization dryer 220 may include the drain line 229 for draining moisture, which is formed at the bottom of the storage space 221. In other words, moisture generated during the sterilization, drying, or deodorization of the clothing or shoes in the storage space 221 may be drained through the drain line 229. Accordingly, the interior of the storage space 221 may remain in a dry state.

Referring to FIGS. 4 and 5, in the drying device for vehicles 200 according to the embodiment of the present disclosure, the air conditioner 210 may include: the exhaust hole 211 connected to the intake line 222 of the sterilization dryer 220 and capable of being opened and closed; and the intake hole 212 connected to the exhaust line 224 of the sterilization dryer 220 and capable of being opened and closed. Furthermore, the air conditioner 210 may include the opening/closing member 213 that controls the opening and closing of the exhaust hole 211 and the intake hole 212.

As described above, the drying device for vehicles 200 according to the embodiment of the present disclosure aims to sterilize, dry, and deodorize the clothing or shoes in the vehicle 100 based on the air conditioning function of the vehicle 100.

Therefore, in addition to performing the conventional air conditioning function in the vehicle 100, the air conditioner 210 may direct air into the sterilization dryer 220 to sterilize, dry, and deodorize the clothing or shoes stored in the storage space 221 based on the input of the occupant received through the user input unit 110 as described in FIGS. 1 and 2.

More specifically, the opening/closing member 213 may have sections capable of being opened and closed as shown in FIG. 5. For example, external air may be drawn in through the opening and closing of a first section 213a, while internal air may be drawn in through the opening and closing of a second section 213b. In other words, the first section 213a and the second section 213b may be designed to facilitate the general air conditioning functions of the vehicle 100.

In the drying device for vehicles 200 according to the embodiment of the present disclosure, the air conditioner 210 may include a third section 213c capable of being opened and closed in the opening/closing member 213. Through the opening and closing of the third section 213c, air may flow between the air conditioner 210 and the sterilization dryer 220.

Additionally, the processor 120 described in FIG. 1 may control the opening and closing of the sections of the opening/closing member 213. The processor 120 may also control the opening and closing of the sections of the opening/closing member 213 based on the input of the occupant received via the user input unit 110.

Referring to FIG. 7, in the drying device for vehicles 200 according to the embodiment of the present disclosure, the air 10 exhausted from the air conditioner 210 may be dry air. The dry air may flow into the storage space 221 through the intake line 222 connected to the exhaust hole 211.

The air that flows into the storage space 221 may become humid due to contact with moisture generated during the sterilization, drying, and deodorization of the clothing or shoes. In other words, the air 20 that passes through the storage space 221 and flows into the exhaust line 224 may be humid air.

The blower 214 described in FIG. 1 may allow the humid air in the storage space 221 to pass through the intake hole 212 connected to the exhaust line 224. Then, the air may flow back into the air conditioner 210.

Thereafter, the air conditioner 210 may dehumidify the air to dry the humid air. The dried air may then flow back into the storage space 221 through the intake line 222 connected to the exhaust hole 211.

As the described process repeats, air circulates between the air conditioner 210 and the sterilization dryer 220. The continuous air circulation may sterilize, dry, and deodorize the clothing or shoes stored in the storage space 221.

The drying device for vehicles 200 according to the embodiment of the present disclosure may also allow external air to be introduced through the intake line 222. The external air entering through the intake line 222 may pass through the dust collection filter 228 and the antimicrobial sheet 227 described in FIG. 8 and then directed into the storage space 221.

Additionally, referring to FIGS. 6 and 7, the drying device for vehicles 200 according to the embodiment of the present disclosure may open and close at least one of the discharge line 223 or the exhaust line 224 to move the air that has passed through the storage space 221. In other words, the drying device for vehicles 200 may discharge the air introduced through the intake line 222 to the outside via the discharge line 223 or flow external air into the air conditioner 210 via the exhaust line 224, thereby allowing the clothing or shoes stored in the storage space 221 to be sterilized, dried, and deodorized.

Furthermore, as described above, the sterilization dryer 220 in the drying device for vehicles 200 according to the embodiment of the present disclosure may be installed in at least one of a floor panel, seat, or trunk. The air conditioner 210 may also be positioned at various locations within the vehicle 100. For example, if the sterilization dryer 220 is installed in the trunk, the sterilization dryer 220 may be connected to an air conditioner 210 located in the trunk. Such arrangement offers the advantage of enhancing space utilization within the vehicle 100.

The block diagrams of the vehicle 100 and the drying device for vehicles 200 shown in FIGS. 1 and 2 are merely block diagrams for an embodiment of the present disclosure. Each component in the block diagrams may be integrated, added, or omitted depending on the specifications of the vehicle 100 and the drying device for vehicles 200 to be implemented.

In other words, depending on the necessity, two or more components may be combined into a single component, or one component may be subdivided into two or more components. Additionally, the functions performed by each block are intended to explain embodiments of the present disclosure, and the specific operations or devices do not limit the scope of the rights of the present disclosure.

In summary, the drying device for vehicles and the vehicle equipped with the same according to the present disclosure may sterilize, dry, and deodorize clothing or shoes inside the vehicle using the air conditioning function of the vehicle. Additionally, the drying device for vehicles and the vehicle equipped with the same may enhance space utilization within the vehicle, thereby storing and managing the clothing or shoes of the occupant efficiently.

The above detailed description is to be construed in all aspects as illustrative and not restrictive. The scope of the present disclosure should be determined by reasonable interpretation of the appended claims and all changes coming within the equivalency range of the present disclosure are intended to be embraced in the scope of the present disclosure.

What is claimed is:

1. A drying device for a vehicle, the drying device comprising:
an air conditioner configured for exhaust and intake; and
a sterilization dryer connected to the air conditioner and having formed therein a storage space,
wherein the sterilization dryer comprises:
an intake line configured to direct air exhausted from the air conditioner into the storage space;
a discharge line configured to externally discharge air that has flowed through the intake line and passed through the storage space; and
an exhaust line configured to direct air that has flowed through the intake line and passed through the storage space, into the air conditioner.

2. The drying device of claim 1, wherein at least one of the discharge line or the exhaust line is opened and closed to move the air that has passed through the storage space.

3. The drying device of claim 1, wherein the sterilization dryer comprises:
a heating member configured to heat the storage space; and
an ultraviolet (UV) light emitting diode (LED) configured to emit UV light into the storage space.

4. The drying device of claim 3, wherein the heating member comprises at least one of a heating coil configured to generate heat based on application of an electric current or a steam generator configured to generate heat by heating fluid.

5. The drying device of claim 1, wherein the sterilization dryer comprises an antimicrobial sheet configured to be detachable from the intake line.

6. The drying device of claim 1, wherein the sterilization dryer comprises a dust collection filter configured to be detachable from at least one of the intake line or the storage space.

7. The drying device of claim 1, wherein the sterilization dryer comprises a drain line formed at a bottom of the storage space and configured to drain moisture.

8. The drying device of claim 1, wherein the sterilization dryer is installed in at least one of a floor panel, a seat, or a trunk, and
wherein a size of the storage space varies depending on the installation location.

9. The drying device of claim 1, wherein the air conditioner comprises:
an exhaust hole connected to the intake line and configured to be opened and closed;
an intake hole connected to the exhaust line and configured to be opened and closed; and
an opening/closing member configured to control opening and closing of the exhaust hole and the intake hole.

10. A vehicle comprising:
a user input unit configured to receive an input from an occupant of the vehicle;
a drying device installed in a body of the vehicle; and
a processor configured to operate the drying device based on the received input from the occupant,
wherein the drying device comprises:
an air conditioner configured for exhaust and intake; and
a sterilization dryer connected to the air conditioner, the sterilization dryer including a storage space,
wherein the sterilization dryer comprises:
an intake line configured to direct air exhausted from the air conditioner into the storage space;
a discharge line configured to externally discharge air that has flowed through the intake line and passed through the storage space; and
an exhaust line configured to direct air that has flowed through the intake line and passed through the storage space, into the air conditioner.

11. The vehicle of claim 10, wherein the sterilization dryer further comprises:
a heating member configured to heat the storage space; and
an ultraviolet (UV) light emitting diode (LED) configured to emit UV light into the storage space and disinfect.

12. The vehicle of claim 10, wherein the heating member comprises a heating coil.

13. The vehicle of claim 10, wherein the sterilization dryer comprises an antimicrobial sheet configured to be detachable from the intake line.

14. The vehicle of claim 10, wherein the sterilization dryer comprises a dust collection filter configured to be detachable from at least one of the intake line or the storage space.

15. The vehicle of claim 10, wherein the sterilization dryer is configured to be installed in at least one of a floor panel, a seat, or a trunk of the vehicle.
